(19) United States
(12) Patent Application Publication
(10) Pub. No.: US 2020/0237914 A1
(43) Pub. Date: Jul. 30, 2020

(54) Throat Lozenge

(71) Applicant: Tazio Stephan Grivetti, Peoria, IL (US)

(72) Inventor: Tazio Stephan Grivetti, Peoria, IL (US)

(21) Appl. No.: 16/748,758

(22) Filed: Jan. 21, 2020

Related U.S. Application Data
(60) Provisional application No. 62/797,996, filed on Jan. 29, 2019.

(51) Int. Cl. A61K 41/00; A61K 9/00

(52) U.S. Cl. CPC A61K 41/0052; A61K 9/0056

(57) ABSTRACT

An assembly of throat lozenge and applicator has improved therapeutic properties, further augmented by following a method consisting of a heating procedure using a flavor or efficacy-enhancing liquid. The throat lozenge is molded onto a distal end of an applicator adapted for holding the throat lozenge at a desired location within the mouth. The lozenge containing a plurality of cavities of specific depth and placement on the upper surface or shell of the lozenge, that following a proscribed method are filled with a therapeutic liquid. The combination of all elements of the invention and following the method results in improved perceived efficacy.

### RELATED APPLICATION

This application claims the priority of provisional patent application Ser. No. 62/797,996 filed Jan. 29, 2019, the contents of which is incorporated herein by reference.

### FIELD OF THE INVENTION

The present invention relates to orally ingestible compositions, including solid compositions as well as liquid-center filled confectionary compositions, for soothing of sore or irritated throats, and relief of congested nasal passages.

### BACKGROUND OF THE INVENTION

Throat lozenges, also known as cough drops, have long been known as vehicles for the delivery of therapeutic agents aimed at soothing sore or irritated throats. Such agents include analgesics, antitussives, expectorants, cooling agents such as menthol, and perceived warming agents such as ethanol or gingerol. The agent can be administered by way of a throat lozenge which releases the active agent by upon application of saliva enzymes in the mouth. In the case of a volatile active agent, the product can also provide relief from cold symptoms by way of clearing the nasal passages. Throat lozenges typically contain a relatively small amount of therapeutic agent in a hard body formed from corn syrup, sugar, flavorings, and dyes. Throat lozenges are typically solids that are ingested at room temperature and are held on the tongue as they dissolve. Throat lozenges are typically homogeneous, but some contain liquid or gel centers inside a hard shell.

U.S. Pat. No. 4,372,942 describes hard candies with a center filled with an additional substance as does U.S. Pat. No. 4,466,983 and such methods are long standing and well known.

EP-A-431,376 describes hard confections for sustained release treatment of sore throats comprising hydrogenated isomaltulose and an active ingredient which can be an antitussive or antihistamine but can also be a volatile oil such as menthol or eucalyptus. The confection normally contains a further flavoring agent such as lemon, cherry, menthol, or eucalyptus.

U.S. Pat. No. 6,432,441 describes hard confections with an aqueous filling, which contains vesicle-forming agent which encapsulates the throat relief agents with vesicles which are dispersed within the filling. The vesicles have a number average particle size of from about 1 to about 100 μm. The vesicles are thought to prolong the duration of the relief provided by the throat drop

U.S. Pat. No. 6,231,900 describes confectionary product preparation, and the placement of flavorings and medicinal components in separate layers in the confection. U.S. Pat. No. 4,980,169 describes a method for flavor enhancing and thereby increasing the efficacy of cough drops. U.S. Pat. No. 6,280,762 describes a throat drop with aqueous filling for medicinal purposes.

However, a demand still exists for an improved throat lozenge.

### SUMMARY OF THE INVENTION

The general object of this invention is to provide an improved therapeutic experience utilizing a novel assembly including an improved throat lozenge, applicator, liquid composition and method for use and consumption.

I have invented an improved throat lozenge assembly and method. The improved lozenge is approximately 2× to 4× larger than the typical throat lozenge. The lozenge is flat on the bottom, with a domed top having a plurality of symmetrical, circular cavities on the upper surface which descend approximately half-way into the body of the lozenge. The lozenge is molded onto a distal end of an applicator adapted for holding the throat lozenge at a desired location within the mouth.

The method utilizes the assembly described, and immerses the distal end (the lozenge) into a heated liquid until the lozenge reaches a temperature (varying according to the wishes of the user) of about 110 to 140° F. The user raises the lozenge from the submersion with the domed side facing upward such that the cavities remain filled with the liquid, which is therapeutic and flavor-matched to the flavor of the lozenge itself. The lozenge is inserted into the oral cavity and placed in the desired position. The flavor and qualities of the lozenge itself combine with the small amount of therapeutic liquid in the cavities (of varying flavors according to the tastes of the user) and the soothing heat to yield an improved and much-prolonged experience versus other methods.

### BRIEF DESCRIPTION OF THE DRAWINGS

FIG. 1 is a perspective view of a throat lozenge of this invention having cavities for holding a therapeutic liquid.

FIG. 2 is a view of a throat lozenge molded onto an applicator, and compared to other lozenges for size and assembly.

FIG. 3 is a view thereof in use including a closeup of the fluid dynamics.

### DETAILED DESCRIPTION OF THE INVENTION

The improved throat lozenge assembly and method of this invention has a preferred embodiment. The lozenge assembly itself can provide relief to users and can be used separately from the described method but is preferred to be used following the prescribed method for maximum efficacy.

The composition of the throat lozenge is conventional. The throat lozenge is generally formed primarily from a solution of corn syrup, sugar, flavorings, and dyes. A small amount of active agent is included as desired. The amount of active agent is typically about 0.01 to 10 percent by weight.

The preferred embodiment of the invention is a throat lozenge having a temperature of about 110 to 140° F. The throat lozenge may be conventional in shape, size, and composition. However, the preferred throat lozenge is modified to better absorb and retain heat, and then transfer that heat to the palate of the user. For example, the heat retention is preferably accomplished by increasing the size of the lozenge, changing the compound of the lozenge, utilizing a solid edible body, with specifically-placed voids or holes in the upper surface of the outer shell, or any combination thereof. The throat lozenge is heated in any known way that does not destroy the lozenge, including heating in a microwave oven, a conventional oven, or by immersing for a short time in a hot liquid, that may or may not contain medicinal properties. The throat lozenge is ingested while in a heated state.

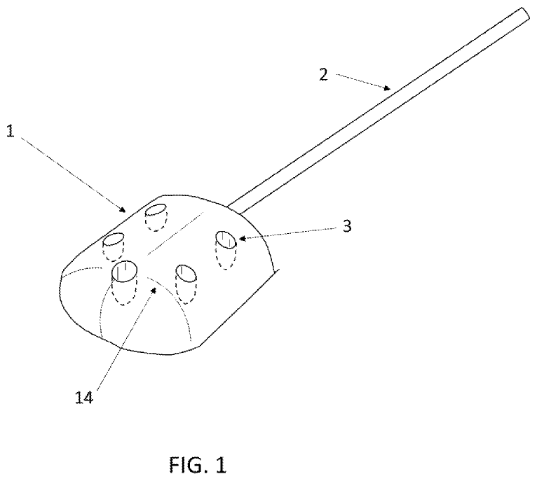

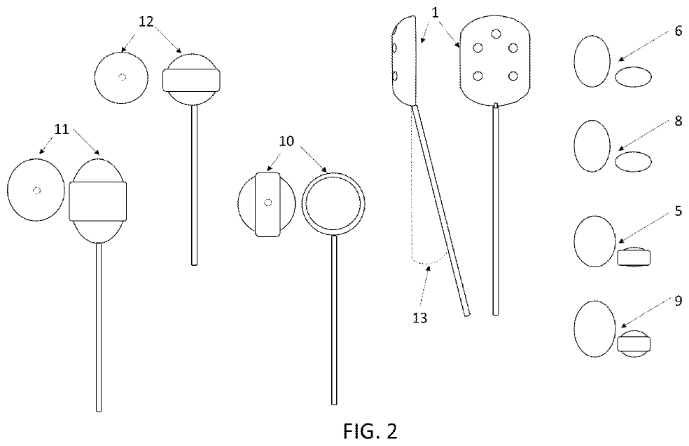

Referring now to FIG. 1, the preferred embodiment of the invention is a throat lozenge (1) with applicator (2) and depicting several engineered cavities (3) on the upper surface. In FIG. 2, the invention (4) is shown to be several times larger than existing throat lozenges with popular trade names such as Halls (5), Ludens (6), Smith Brothers (7), Ricola (8), and Cold-EEZ (9). There are numerous other similar products but all are close to the size of those depicted. Each of these comparator throat lozenges are symmetrical in shape, do not have an applicator, and do not have integrated holes or cavities in the outer shell. The size difference compared to current popular lozenges is shown below:

| Feature | Ludens | Thermal Lozenge | % size |
|---|---|---|---|
| Max Length | 23.3 mm (.92 in) | 37.7 mm (1.48 in) | 161%+ |
| Max Width | 15.4 mm (.6 in) | 24.7 mm (.97 in) | 160%+ |
| Max Height | 9.7 mm (.38 in) | 12.5 mm (.5 in) | 128%+ |
| Stick Length | N/A | 100+ mm (4+ in) | |

| Feature | Ricola | Thermal Lozenge | % size |
|---|---|---|---|
| Max Length | 24.3 mm (.95 in) | 37.7 mm (1.48 in) | 155%+ |
| Max Width | 15.2 mm (.6 in) | 24.7 mm (.97 in) | 162%+ |
| Max Height | 9.7 mm (.38 in) | 12.5 mm (.49 in) | 128%+ |
| Stick Length | N/A | 100+ mm (4+ in) | |

| Feature | Halls | Thermal Lozenge | % size |
|---|---|---|---|
| Max Length | 21.2 mm (.83 in) | 37.7 mm (1.48 in) | 177%+ |
| Max Width | 15.8 mm (.62 in) | 24.7 mm (.97 in) | 156%+ |
| Max Height | 9.4 mm (.37 in) | 12.5 mm (.49 in) | 133%+ |
| Stick Length | N/A | 100+ mm (4+ in) | |

| Feature | Cold-Eeze | Thermal Lozenge | % size |
|---|---|---|---|
| Max Length | 22.4 mm (.88 in) | 37.7 mm (1.48 in) | 168%+ |
| Max Width | 16.4 mm (.64 in) | 24.7 mm (.97 in) | 150%+ |
| Max Height | 13 mm (.5 in) | 12.5 mm (.49 in) | ~same |
| Stick Length | N/A | 100+ mm (4+in) | |

In FIG. 2, the invention is also compared to existing sucker or lollipop brands such as Tootsie-Pop (10), Charms Blow-Pop (11) and Dum Dums (12) which are generally consumed for the flavor and have no medicinal or therapeutic properties. They are all symmetrical in shape, have a “stick.” The larger suckers are closer in size to the invention, but they all lack the holes to retain liquid in the outer, upper shell. In fact, they are all symmetrical and do not have an upper and lower surface designed to fit into the human palate for best heat transfer. In addition, and the applicator of the invention is much longer than the “stick” protruding from the suckers. The stick in the suckers is always inserted into the center of a spherical object. This results in sub-optimal placement wherein to clear the teeth, the sucker is a poor angle where the sphere is only in contact with the palate at a tangential basis. With the invention, the applicator is inserted into the body of the invention lozenge at an angle (13) that facilitates optimum placement of the lozenge against the roof of the mouth or palate. Likewise, the curve of the invention at the forefront, combined with a flatter portion in the middle upper surface allows for more surface area contact.

| Feature | Dum Dum | Thermal Lozenge | % size |
|---|---|---|---|
| Max Length | 20.2 mm (.79 in) | 37.7 mm (1.48 in) | 186%+ |
| Max Width | 20.8 mm (.82 in) | 24.7 mm (.97 in) | 18%+ |
| Max Height | 20.2 mm (.79 in) | 12.5 mm (.49 in) | Smaller |
| Stick Length | 64.4 mm (2.53 in) | 100+ mm (4+ in) | 155%+ |

| Feature | Charms | Thermal Lozenge | % size |
|---|---|---|---|
| Max Length | 33.6 mm (1.32 in) | 37.7 mm (1.48 in) | 12%+ |
| Max Width | 26.6 mm (1.04 in) | 24.7 mm (.97 in) | Smaller |
| Max Height | 26.6 mm (1.04 in) | 12.5 mm (.49 in) | Smaller |
| Stick Length | 69.4 mm (2.73 in) | 100+ mm (4+ in) | 144%+ |

| Feature | Tootsie Pop | Thermal Lozenge | % size |
|---|---|---|---|
| Max Length | 27 mm (1.06 in) | 37.7 mm (1.48 in) | 139%+ |
| Max Width | 26.6 mm (1.04 in) | 24.7 mm (.97 in) | Smaller |
| Max Height | 26.6 mm (1.04 in) | 12.5 mm (.49 in) | Smaller |
| Stick Length | 69.4 mm (2.73 in) | 100+ mm (4+in) | 144%+ |

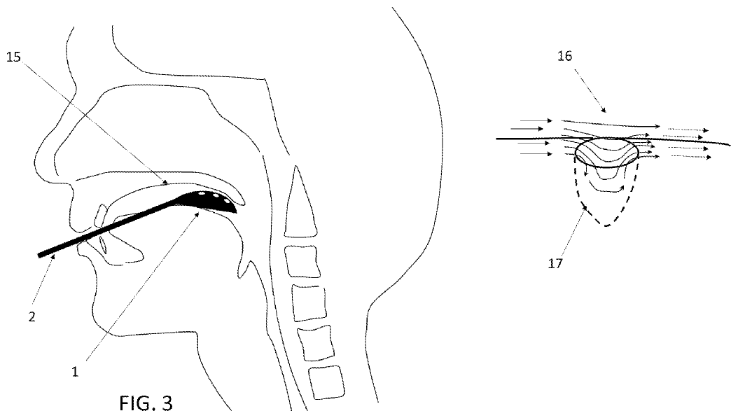

Referring now to FIGS. 2 and 3, the preferred embodiment of the invention is an assembly of a throat lozenge (1) with integrated holes or voids (3) on the outer shell, molded onto a distal end of an applicator (2). This shape and angle (13) of the applicator is especially adapted for holding the throat lozenge at a desired location within the mouth or the roof of the mouth, while clearing the teeth when inserted. The integral applicator allows the throat lozenge to be held against the back of the roof the mouth where the tongue is unable to reach. The applicator is disposable after use and is typically made of thermoplastic or paper. The shape of the lozenge is specifically dome-shaped (14) to better match the contours of the roof of the mouth (15), for optimum heat-transfer.

The placement of the cavities or voids (3) on the upper surface of the lozenge and the depth of the voids is specifically engineered. A plurality of voids spaced along the upper shell or roof of the lozenge is advantageous, but must avoid the rear portion of the lozenge where the applicator is inserted to allow the lozenge body to remain attached to the applicator even as it slowly dissolves during use. The method of use has the user submerging the lozenge in a liquid composition with flavorings and/or medicinal properties, heated according to the preferences of the user. When the lozenge is placed in the mouth, the user typically gently sucks on the lozenge.

The fluid dynamics of the sucking action causes saliva to travel around the body of the sucker on all sides, always toward the rear of the mouth where it is swallowed. As saliva travels along the upper shell of the lozenge, it mixes (16) with the liquid present in the cavities of the lozenge. Through the natural process of osmosis, coupled with the fluid dynamics of the traveling saliva, some of the flavored and/or medicinal liquid is 140 intermixed with saliva and travels rearward into the mouth where the taste buds sense the enhanced flavor as the user swallows. The user can re-insert the lozenge into the liquid composition as many times as is desired, to re-enhance the flavor and therapeutic effect. The liquid composition can be re-heated in a microwave oven to maintain the desired temperature to maintain the therapeutic effect of the heated lozenge. The therapeutic effect of the combined features results in a greatly enhanced experience by the user, versus existing smaller lozenges, lozenges not optimally designed for heat transfer, unheated lozenges, lozenges without precision applicators, and lozenges without engineered cavities for holding liquid.

### EXAMPLE

Tests were conducted by using confections heated to a temperature of 120 to 130° F. in the following categories: (A) confections of average size with a solid consistency; (B) confections of average size with a hard edible outer shell with aqueous filling; (C) confections having a size double the average existing confection size; and (D) confections with cavities formed in the upper surface of the specimen for the purpose of 155 containing a medicinal aqueous component. A control group and study group were used in all cases. It was found in all cases that the perceived efficacy of the heated lozenge was noticeably improved over the unheated lozenge, and following the prescribed method of immersing the lozenge with engineered cavities into a flavor enhanced, heated liquid before ingestion resulted in the greatest perceived efficacy.

I claim:

1. An assembly featuring a throat lozenge having a temperature of about 110 to 140° F., having engineered cavities on the upper surface of the outer shell or body, which is molded onto the distal end of an applicator adapted for holding the throat lozenge at a desired location within the mouth.

2. A method according to claim 1 wherein the user immerses the assembly into a heated flavor-enhancing or efficacy-enhancing liquid prior to ingestion which serves to keep the assembly in a heated state, and allows repeated delivery of minute amounts of liquid via the cavities of the lozenge assembly of claim 1.
